(19) United States
(12) Patent Application Publication
(10) Pub. No.: US 2009/0081768 A1
(43) Pub. Date: Mar. 26, 2009

(54) Devices and Methods for Thermally Isolating Chambers of an Assay Card

(75) Inventors: Joon Mo Yang, Redwood City, CA (US); David M. Liu, Los Altos, CA (US); Yuh-Min Chiang, Foster City, CA (US); Carol Schrembri, San Mateo, CA (US); Aldrich N.K. Lau, Palo Alto, CA (US); Umberto Ulmanella, Foster City, CA (US); Nigel P. Beard, Redwood City, CA (US); Maryam Shariati, Sunnyvale, CA (US); James C. Nurse, Pleasanton, CA (US); Douwe D. Haga, Redwood City, CA (US); Ian A. Harding, San Mateo, CA (US); Julio P. Focaracci, San Mateo, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 11/859,180

(22) Filed: Sep. 21, 2007

(51) Int. Cl. C12M 1/34; B29C 59/02; B29C 59/16

(52) U.S. Cl. 435/288.4; 264/293; 264/442; 264/101

(57) ABSTRACT

An assay card and devices and methods for isolating chambers on the assay card are described. The assay card comprises a substrate formed of one or more materials, e.g., plastic, having a softening temperature, the substrate defining channels communicating with respective reaction chambers. The assay card may be heated in a region of the channels to at least the softening temperature. The softened plastic may be deformed, e.g., with a tool which may or may not also provide the heat for softening the substrate. In this manner, the plastic of the substrate may be caused to at least partially obstruct the channels, thereby isolating the reaction chambers. The invention also relates to a method of manufacturing a tool device that includes pins for heating and deforming an assay card.

### GOVERNMENT SUPPORT

The present invention may have been made with support from the U.S. Government under United States Air Force Contract No. FA7014-06-C-0017. The U.S. Government may have certain rights in the inventions recited herein.

### FIELD OF THE INVENTION

The invention relates to devices and methods for isolating chambers of an assay card, and more particularly to devices and methods for isolating chambers of an assay card by softening and/or deforming at least a portion of the assay card. The invention also relates to a method of manufacturing a tool device that includes one or more tools for heating and deforming portions of an assay card.

### BACKGROUND

The detection and monitoring of diseases commonly employs various types of biological testing. Since there are often a large number of tests that are required to be performed in this field, it is typically desirable to reduce the cost and time associated with these tests. A commonly employed technique for reducing such costs and time is to simultaneously test numerous, relatively small samples, e.g., during each run of a thermal cycling unit or other like device. Substrates having multiple wells, detection chambers or reaction chambers to which a fluid sample is distributed through one or more channels to which the chambers are connected have been employed for simultaneously testing a large number of analytes in such a sample. Such substrates, which are sometimes referred to as “microcards,” “assay cards” or “analytical cartridges,” allow relatively small sample volumes to be distributed to a large number of detection chambers, such as 96, 384, or more, which may be preloaded with different analyte-specific reagents. Such substrates, in addition to systems and method for their use, are described, for example, in U.S. Pat. No. 6,126,899, U.S. Pat. No. 6,272,939 and U.S. Patent Publication No. 2004/0157343.

In substrates in which channels interconnect the chambers or detection chambers there is a potential for fluid communication between chambers during sample processing, for example, during thermal cycling, which may cross-contaminate the reactions in the connected chambers, Various strategies are disclosed in the art to reduce the potential for cross-contamination. For example, U.S. Pat. No. 6,126,899 discloses filling the delivery channels with an additional fluid, such as a mineral oil or a viscous polymer solution, to segregate the chambers from each other. U.S. Pat. No. 6,068,751 discloses the use of a valve between processing chambers that is closed to isolate the processing chambers from each other. U.S. Patent Publication No. 2004/0157343 discloses sealing of each loading passage connecting each of a series of chambers to a common channel by deforming the substrate cover adjacent each loading passage. The cover is deformed by bringing the substrate into contact with a thermal transfer block having bosses or protrusions in locations corresponding to the loading passages. The bosses can be heated to facilitate deformation of the cover material.

However, these and other devices and method for preventing contamination between chambers or detection chambers, may not do so in a manner that is adequately safe, reliable, and fast. In light of the foregoing, there is a need for a system and method that overcomes the disadvantages of the previous methods.

### SUMMARY

The invention relates to an assay card and a method for isolating chambers on an assay card. In this embodiment, the assay card comprises a substrate formed of plastic having a softening temperature, the substrate defining a first channel in communication with a first chamber and a second channel in communication with a second chamber. The method comprises the steps of heating the assay card in a region of the first and second channels to at least the softening temperature; and simultaneously deforming, with a single tool, the assay card in the region of the first and second channels such that plastic of the substrate is caused to at least partially obstruct both the first and second channels. The method may also include the step of cooling the deformed plastic.

In an embodiment, the heating step includes contacting the substrate with a heated tool. Contacting the substrate with a heated tool may also include inserting the tool into the channel, and/or contacting a region of the substrate adjacent to the channel. The heating step may include applying a source of ultrasonic energy to the substrate. Alternatively, the heating step may include directing a light or laser beam or a heated air jet to the substrate.

Additionally or alternatively to the substrate being deformed by contact and/or pressure with a tool, the deforming step may also occur spontaneously due to the surface tension of the softened region of the substrate. In the same manner that a water droplet may spontaneously flow into a capillary because of the surface tension, material from the softened region of the substrate may spontaneously flow into the channel without the application of a mechanical tool. Channels with properly designed features in the softened region of the substrate will allow the surface tension to locally pull the material from the substrate to obstruct the channel and to automatically cease the filling action after the channel is completely obstructed. After the substrate material cools down, the channel is thus permanently obstructed. An advantage of this technique is that it employs a passive mechanism, hence not requiring the mechanical application of a tool.

Also, the deforming step may include permitting gravity to deform the softened region of the substrate. Furthermore, the deforming step may include the application of a pneumatic pressure or vacuum to the substrate. Still further, the deforming step may include moving the assay card so as to cause an inertial stress onto the substrate, which in turn will deform and flow to locally fill the channel. In the same manner that a surface of a liquid within a container may change its shape when the container is set in motion, the softened region of the substrate may be made to flow into the channel by subjecting the card to a motion that would cause inertial forces and thus stresses, such as a rotary motion (centrifuge) or an impulsive linear motion (slide).

Various spatial arrangements of the substrate and, e.g., the tools, may be possible. For example, the heating and deforming steps may include heating and deforming the assay card from a side of the substrate to which the channels are directly or most nearly adjacent. Additionally or alternatively, the heating and deforming steps may include heating and deforming the assay card from a side of the substrate that is opposite the side of the substrate to which the channels are directly or most nearly adjacent.

The invention, in accordance with another embodiment thereof, relates to a method for isolating a reaction chamber of an assay card. In this embodiment, the assay card comprises a substrate formed of plastic having a softening temperature, the substrate having a first surface and a second surface opposite the first surface, the substrate defining the chamber, the substrate further defining a channel adjacent the first surface and in communication with the chamber, the substrate further defining in the second surface a depression that is aligned with at least a portion of the channel. The chamber and the channel may be sealed by a second layer attached to the first surface of the substrate. The method may comprise the steps of: heating the assay card in a region of the depression and the channel to at least the softening temperature; and deforming the assay card in the region of the depression such that plastic of the substrate is caused to at least partially obstruct the channel. The method may also include the step of cooling the deformed plastic.

Also, the heating and deforming steps may include contacting and applying pressure to a surface of the depression of the substrate with a heated tool. Additionally or alternatively, the heating step may include applying a source of ultrasonic energy, a light or laser beam or a heated air jet to a surface of the depression in the substrate. In an embodiment, the substrate defines two or more chambers and two or more channels, each chamber having a respective channel in communication with it, and wherein the heating and deforming steps simultaneously heat and deform the assay card in a region that includes the two or more channels. The region that includes the two or more channels may be simultaneously heated by contact with a single heated tool.

The invention, in accordance with another embodiment thereof, relates to a method for isolating a chamber of an assay card. In this embodiment, the assay card comprises a substrate formed of a first material having a first softening temperature and a second material having a second softening temperature, the substrate defining a channel in communication with a chamber, the second material being adjacent to the channel. The method comprises the steps of: heating the assay card in a region of the channel to at least the second softening temperature; and deforming the assay card in the region of the channel such that the second material at least partially obstructs the channel.

The first and second materials may be first and second types or grades of plastic, wherein the first softening temperature is greater than the second softening temperature. The heating step may include heating the assay card in a region of the channel to a temperature that is greater than the second softening temperature but less than the first softening temperature. Also, the channel may be located adjacent to a first surface of the substrate, a second surface of the substrate being opposite the first surface and including a depression, wherein the depression is aligned with at least a portion of the channel. Advantageously, the second material may be disposed between respective bottom surfaces of the depression and the channel, such that, upon the second material being heated to at least the second softening temperature, the second material may be deformed to obstruct the channel. In an embodiment, the method may further comprise the step of cooling the deformed plastic. Also, the heating and deforming steps may include contacting and applying pressure to a surface of the depression of the substrate with a heated tool. Alternatively, the second material may be a thin section bonded to the first surface of the substrate to complete the formation of the channel. In this embodiment, the heating and deforming step may including contacting the second material over the channel with a heated source causing the second material to at least partially obstruct the channel.

The invention, in accordance with another embodiment thereof, relates to an assay card. The assay card may include a substrate formed of plastic having a softening temperature, the substrate having a first surface and a second surface opposite the first surface. The substrate may define a chamber, the substrate further defining a channel adjacent the first surface and in communication with the chamber. The chamber and the channel may be sealed by a second layer attached to the first surface of the substrate. The substrate may also define in the second surface a depression that is aligned with at least a portion of the channel, wherein, upon heating the assay card in a region of the depression and the channel to at least the softening temperature, the assay card in the region of the depression is configured to be deformed such that plastic of the substrate may at least partially obstruct the channel. The deformed plastic may be cooled so as to maintain isolation of the chamber.

The assay card may be configured to be deformed by heat and pressure applied by a heated tool. Additionally or alternatively, the substrate may be configured to be heated by the application of a source of ultrasonic energy, a light or laser beam or a heated air jet. The substrate may define two or more chambers and two or more channels, each chamber having a respective channel in communication with it, and the two or more channels may be arranged so as to be simultaneously heated and deformed. The two or more channels may be arranged so as to be simultaneously heated by contact with a single heated tool. The plastic may include a first region having a first softening temperature and a second region having a second softening temperature, wherein the first softening temperature is greater than the second softening temperature. The assay card may be configured to be heated in a region of the channel to a temperature that is greater than the second softening temperature but less than the first softening temperature. In an embodiment, the second region of the plastic may be disposed between the depression and the channel, such that the second region is configured to be heated to at least the second softening temperature and to be deformable for obstructing the channel.

The invention, in accordance with still another embodiment thereof relates to a method of manufacturing a tool device, the tool device including pins for heating and deforming an assay card. The method may comprise the steps of providing a rigid insulator defining through-holes; applying a resist layer to the insulator, wherein the resist layer is patterned with holes which match the through-holes of the insulator; plating pins through the resist layer and the rigid insulator; stripping the resist layer to expose the pins; and forming a conductive pathway linking the pins. The method may also include the step of smoothing and rounding the pins, e.g., by performing an isotropic wet etch process. Also, the step of forming a conductive pathway linking the pins may include patterning a metal layer on a back side of the tool device.

Additional features of the device and methods of the invention are discussed in greater detail below.

### BRIEF DESCRIPTION OF THE DRAWINGS

FIG. 1 is a top view of an assay card, according to one embodiment of the invention;

FIG. 2 is a side cross-sectional view of an assay card, according to one embodiment of the invention;

FIG. 3 is an exploded perspective view of an assay card, according to the embodiment of the invention shown in FIG. 1;

FIG. 4(a) is an assembled top view of the assay card, according to an embodiment of the invention;

FIG. 4(b) is an assembled perspective view of the assay card, according to an embodiment of the invention;

FIGS. 5(a) through 5(d) illustrate the steps that may be performed, according to an embodiment of the invention, to isolate the reaction chambers of an assay card;

FIGS. 6(a) through 6(d) illustrate schematically a method of isolating a reaction chamber by heating and deforming an assay card with a heated tool from a side of the substrate to which channels are directly or most nearly adjacent, according to an embodiment of the invention;

FIG. 7 is a side view that illustrates schematically a method of applying heat to the substrate by applying a source of ultrasonic energy, according to an embodiment of the invention;

FIG. 8 illustrates an assay card being heated by a radiation energy source, according to an embodiment of the invention;

FIGS. 9(a) through 9(d) illustrate schematically a method of isolating a reaction chamber by heating and deforming an assay card with a heated tool from a side of the substrate that is opposite the side of the substrate to which channels are directly or most nearly adjacent, according to an embodiment of the invention;

FIGS. 10(a) through 10(d) illustrate schematically a method of isolating a reaction chamber by heating and deforming an assay card with a heated tool in a region of the substrate that is not aligned with a channel to be obstructed, according to an embodiment of the invention;

FIGS. 11(a) and (b) illustrate an arrangement in which each one of one or more tools may be employed to heat and deform a region of the substrate that includes two or more channels, according to an embodiment of the invention;

FIG. 12 is a side view that illustrates schematically a method of applying heat to the substrate with a heated tool, wherein the substrate is formed of more than one type or grade of plastic, according to an embodiment of the invention;

FIG. 13 is an exploded perspective view of a system that includes an assay card, according to an embodiment of the invention;

FIG. 14 is a flowchart that illustrates the steps that may be employed to manufacture or fabricate a tool device, according to an embodiment of the invention;

FIGS. 15(a) and (b) are perspective top and bottom views, respectively, of a tool device, formed in accordance with the steps set forth in the flowchart of FIG. 14;

FIGS. 16(a) through 16(d) are side cross-sectional views, and FIG. 16(e) is a bottom view, that collectively illustrate a tool device as formed in accordance with the steps set forth in the flowchart of FIG. 14; and

FIGS. 17(a) and (b) are graphs that provide test results, namely Delta Rn curves, for illustrating the effect on temperatures within a reaction chamber when the method of the invention, in accordance with an embodiment, is employed.

FIG. 18 is a graph that provides test results, namely Ct values, for illustrating the PCR of Flu A with various concentrations. Sample was introduced into 24-well assay card and the wells were thermally isolated. PCRs of the same sample in 384-well plate and in assay card by pin staking were performed for control and for comparison.

FIGS. 19(a) and (b) illustrate the PCR of Flu B with a concentration of 1 k copies/μL in an assay card spotted with two different assays—Flu B and Mycoplasma pneumoniae. FIG. 19(a) illustrates the spotting pattern in the 24 wells. FIG. 19(b) shows the Delta Rn curves from PCR of 1 k copies/mL Flu B sample with thermal isolation of the 24 wells. Identical assay card was filled and the wells were isolated by pin staking for control.

### DETAILED DESCRIPTION

The invention, according to various embodiments thereof, is directed to devices and methods for isolating reaction chambers of an assay card.

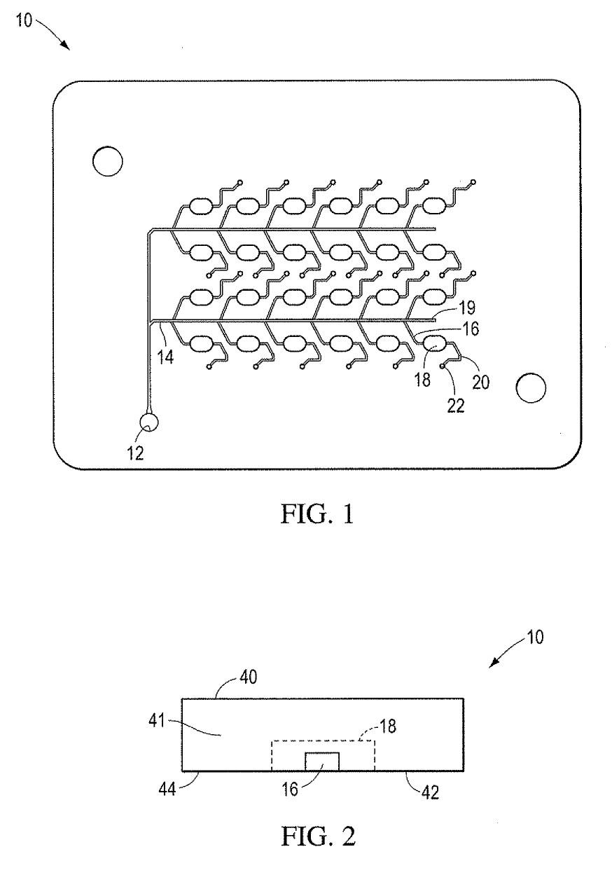

FIG. 1 is a top view of an assay card 10, according to an embodiment of the invention. The assay card 10 may include one or more reaction chambers 18. The assay card 10 may be configured so as to have any number and arrangement of reaction chambers 18. In an embodiment, the assay card 10 may typically contain 96, 384, or more, individual reaction chambers, each typically having a volume of about 1.0 mμL or less in a card size of, for example, 7 cm×11 cm×0.2 cm. In the example shown in FIG. 1, the card size may be 1.5″ (38.1 mm)×2.0″ (50.8 mm)×1 mm. The number of reaction chambers 18 in the assay card 10 may vary anywhere from, for example, one to several thousands, and the individual reaction chamber volume may vary from, for example, 0.001 μL to 1000 mL. The card size may vary from about 1 cm×1 cm to about 25 cm×25 cm.

The assay card 10 may include an inlet port 12 through which a sample is introduced. A bus channel 14 extends from the inlet port 12. Feeder channels 16 branch off of the bus channel 14 and lead into reaction chambers 18. Vent channels 20 extend from the reaction chambers 18 to a vent port 22. In the example embodiment shown in FIG. 1, the bus channel 14 extends across a substantial portion of the width of the assay card 10, and has branch points 19 from which one or more of the feeder channels 16 branch off towards respective reaction chambers 18. It should be understood that any arrangement of channels and ports may be employed. For example, each chamber may have an independent channel from the sample entry point and no bus channel is present. Alternatively, one chamber may be linked directly to another chamber by a fluidic channel.

In addition to the varied arrangement of the reaction chambers, channels and ports, the assay card 10, according to various embodiments of the invention, may be formed of various different layers. For example, FIG. 2 is a side cross-sectional view of an assay card 10, according to an embodiment of the invention. The assay card 10 includes a substrate 41, formed of, for example, a cyclic olefin polymer (COP), having a first surface 40 and a second surface 42 that is opposite the first surface 40. The assay card 10 also includes a pressure sensitive adhesive (PSA) lined metallic foil layer 44 adhered to its second surface 42, the foil layer 44 being formed of, for example, aluminum. The channel 16 is defined within the substrate 41 of the assay card 10, the channel 16 being adjacent to the second surface 42. The channel 16 is in communication with a respective reaction chamber 18. Alternatively, the assay card 10 may be formed from polymethylmethacrylate, polystyrene, polypropylene, polyethylene or other plastics. The channel may be sealed with foil or plastic sheet lined with PSA. It may be sealed with another layer of formed or molded plastic by means of ultrasonic welding, heat lamination, solvent bonding and other means known in the art.

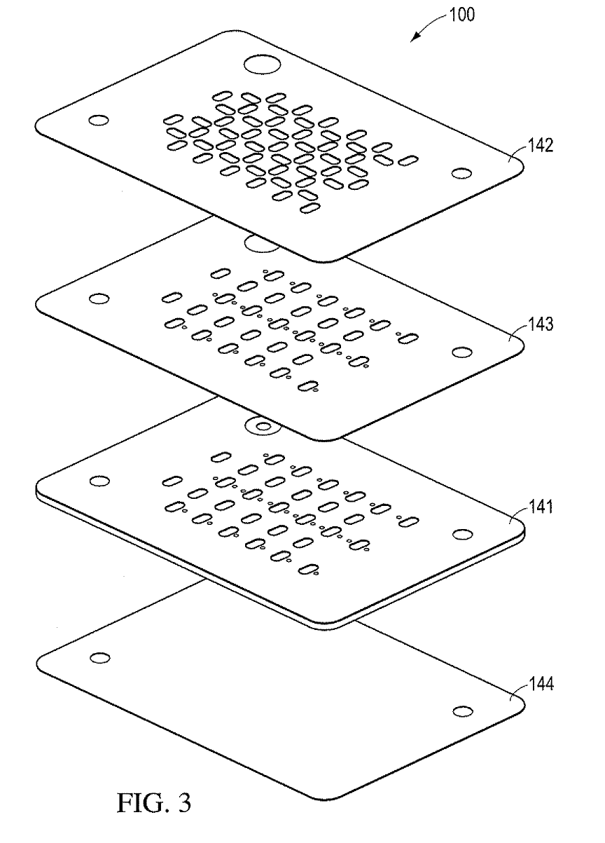

FIG. 3 is an exploded perspective view of an assay card 100, according to another embodiment of the invention. More specifically, FIG. 3 illustrates additional components of an assay card assembly 100. As shown, assay card assembly 100 includes a gas permeable membrane 142. In an embodiment, the gas permeable membrane 142 is hydrophobic. Adjacent to the gas permeable membrane 142 is a PSA lined film 143. In an embodiment, the PSA lined film 143 is hydrophobic. In addition, the PSA lined film may be lined on both sides, Adjacent to the PSA lined film 143 is a substrate 141 formed of, for example, COP. Adjacent to the substrate 141 is a PSA lined film 144 which may be, for example, an aluminum foil. The substrate 141 may have an arrangement of channels that are defined thereby, and the gas permeable membrane 142 and the PSA lined film 143 may have one or more openings that align with respective openings of each other and with portions of the substrate 141, as will be set forth in further detail below.

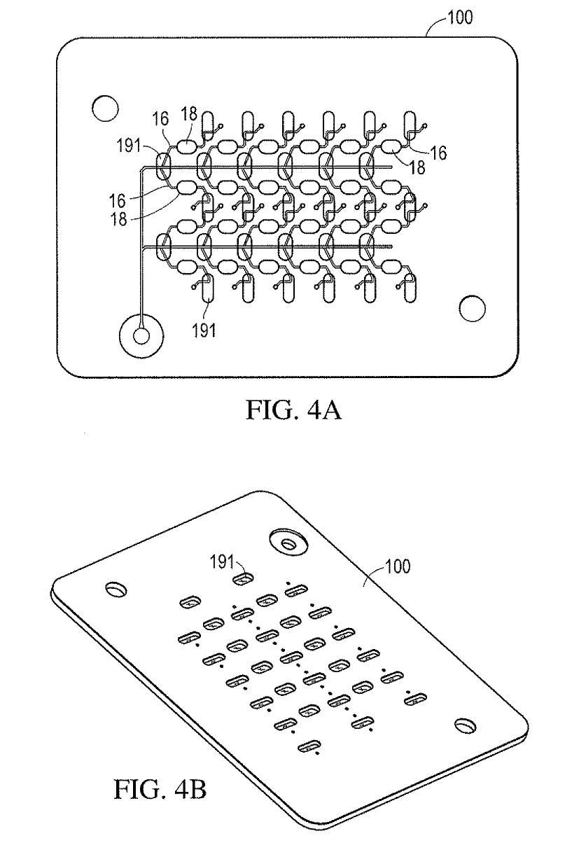

FIGS. 4(a) and 4(b) provide additional views of the assay card 100. Specifically, FIG. 4(a) is an assembled top view of the assay card 100, while FIG. 4(b) is an assembled perspective view of the assay card 100, according to an embodiment of the invention. FIG. 4(a) illustrates an arrangement in which the openings of the gas permeable membrane 142 and the PSA lined film 143, respectively, may align with each other. In addition, FIG. 4(a) illustrates an arrangement in which the openings of the gas permeable membrane 142 and the PSA lined film 143, respectively, may align with the various ports, channels and chambers of the substrate 141. FIG. 4(b) provides a hidden view that illustrates that when the gas permeable membrane 142, the PSA lined film 143, the substrate 141 and the PSA lined film 144 are aligned and assembled to form the assay card 100, the openings may form depressions 191, the arrangement and purpose of which are set forth in additional detail below. Although the assay card 100, and the various layers and features thereof, may have any suitable thickness or depth, in an embodiment, the assay card 100 may have a thickness of about 1 mm, while the depth of the reaction chambers 18 may be about 500 to 700 μm, the depth of the channels 16 may be about 60 μm, and the thickness of the assay card 100 between the respective oppositely-disposed bottom surfaces of the depressions 191 and channels 16 may be about 240 to 440 μm.

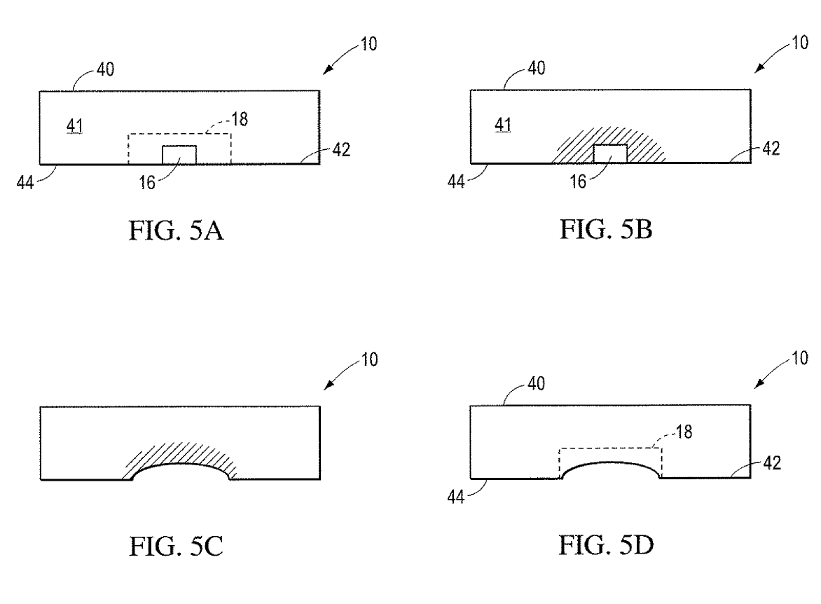

The invention also includes, according to an embodiment, a method to isolate the reaction chambers of an assay card. FIGS. 5(a) through 5(d) illustrate the steps that may be performed, according to an embodiment of the invention, to isolate the reaction chambers 18 of the assay card 10. For example, FIG. 5(a) illustrates the assay card 10, having the channel 16 in communication with the reaction chamber 18. FIG. 5(b) illustrates the assay card 10 being heated in a region of the channel 16 to a predetermined temperature, which advantageously is at least a softening temperature of the substrate 41 in the region of the channel. There are various ways in which the assay card 10 may be heated in the region of the channel 16 to a predetermined temperature, some of which are described in further detail below. FIG. 5(c) illustrates the assay card 10 having been deformed. There are various ways in which the assay card 10 may be deformed in the region of the channel 16, some of which are described in further detail below. The assay card 10 is deformed such that plastic of the substrate 41 is caused to at least partially obstruct the channel 16. Advantageously, the assay card 10 is deformed such that plastic of the substrate 41 is caused to fully obstruct the channel 16, thereby isolating the reaction chamber 18 to which the channel 16 corresponds. In some embodiments, the deformed plastic may be cooled so as to retain its deformed shape so as to continue obstructing the channel 16 such that the reaction chamber 18 remains isolated, as illustrated for example in FIG. 5(d). Alternatively, layer 44 attached to substrate 41 may be comprised of a temperature softening plastic which is heated by a heat source and distorts into channel 16 to obstruct the channel.

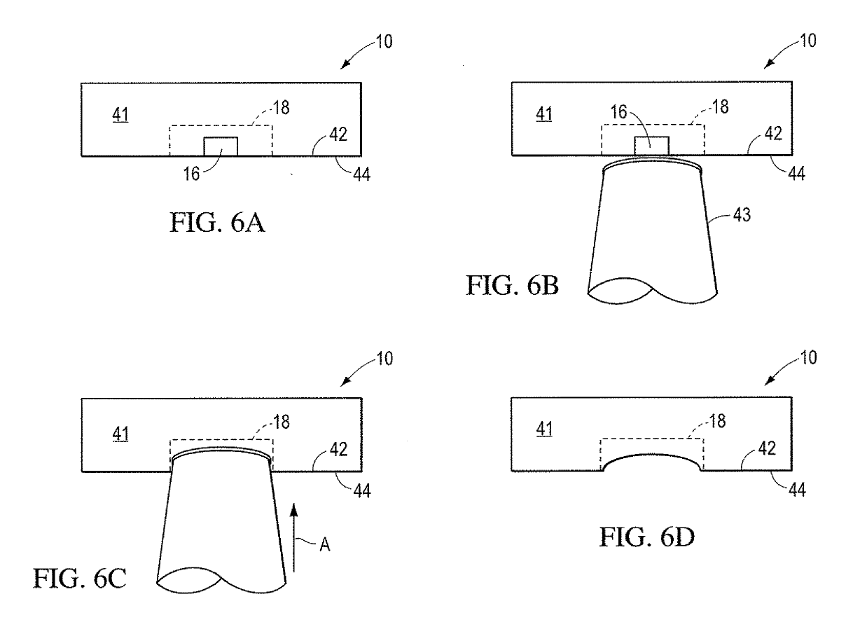

As set forth above, there are various ways in which the assay card 10 may be heated in the region of the channel 16 to a predetermined temperature. For example, the heating step may include contacting the substrate 41 with a heated tool. FIGS. 6(a) through 6(d) illustrate schematically a method of isolating a reaction chamber 18 by heating and deforming an assay card 10 with a heated tool. For example, FIG. 6(a) illustrates the assay card 10, having the channel 16 in communication with the reaction chamber 18. FIG. 6(b) illustrates the assay card 10 being heated by a tool 43 being in contact with the assay card 10. Alternatively, the tool 43 may heat the assay card 10 without actually touching the assay card 10. In an embodiment, the heated tool 43 may be heated to a temperature of about 150 to 250° C., for example to heat a COP substrate 141 that has a softening temperature of 136° C. As will be understood by those skilled in the art, different grades of COP and different plastics have different softening points. The temperature may be optimized for each application.

The tool 43 may be any suitable shape or size, some of which are described in further detail below. Also, the temperature at which the tool 43 may be heated, the duration of contact between the tool 43 and the assay card 10, and the amount of pressure applied may be predetermined or determined by an operator during the heating process. Advantageously, any or all of the factors of tool shape and size, temperature, duration and applied pressure may be varied to insure that the assay card 10, at least in a region of the channel 16, is heated to a predetermined temperature, e.g., at least a softening temperature of the substrate 41 in the region of the channel 16. The heating of the assay card 10 in this manner may provide that the region of the channel 16 is more easily deformed than would be the case had the region not been heated.

FIG. 6(c) illustrates the assay card 10 being deformed by the tool 43 applying a pressure to the assay card 10. In the embodiment shown, the tool 43 is positioned directly over the channel 16, and the motion of the tool 43 (shown as arrow A) is perpendicular to the surface 42 of the assay card 10. It should be recognized that the manner in which the assay card 10 may be deformed in the region of the channel 16 may be varied by varying the position of contact of the tool 43 relative to the channel 16, the direction of motion of the tool, the amount of pressure applied, and other factors. In the embodiment shown in FIG. 6(c), the assay card 10 is deformed such that plastic of the substrate 41 fully obstructs the channel 16. As shown in FIG. 6(d), the deformed plastic of the substrate 41 may be allowed to cool such that the obstruction of the channel 16 is maintained.

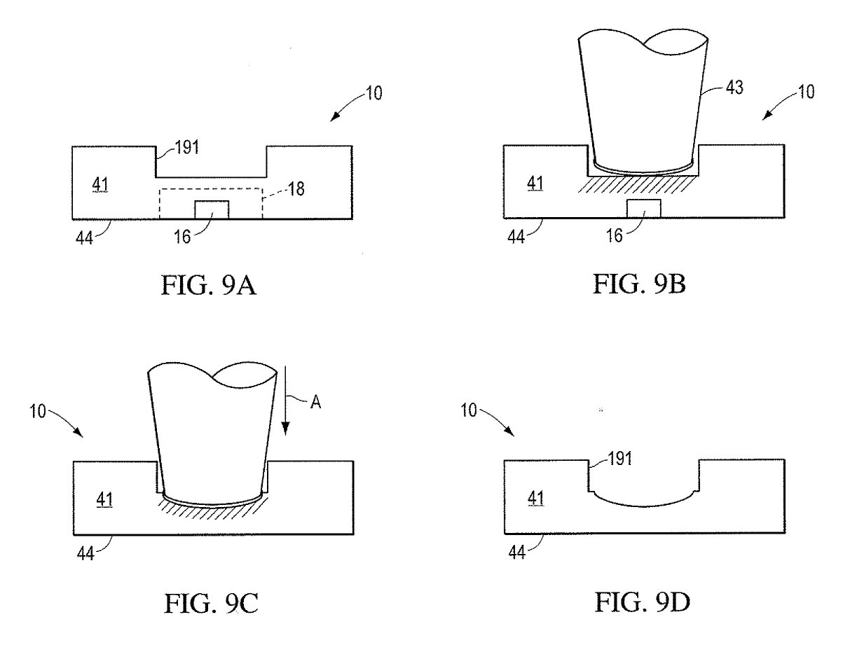

Again, it should also be recognized that the relative positions of the heat source or tool that heats the assay card 10 may be varied in the invention, depending on a number of factors. For example, in an embodiment, the heat source or tool acts on a first surface of the substrate that is opposite the surface that is directly or most nearly adjacent to the channel 16 (which may be the top or the bottom surface depending on the location of the channel). FIGS. 9(a) through 9(d) illustrate schematically a method of isolating a reaction chamber 18 by heating and deforming an assay card 10 with a heated tool applied to a first surface of the substrate that is opposite the surface that is directly or most nearly adjacent to the channel 16. For example, FIG. 9(a) illustrates the assay card 10, having the channel 16 in communication with the reaction chamber 18, wherein the channel 16 is located adjacent to a bottom surface of the assay card 10. FIG. 9(b) illustrates the assay card 10 being heated by a tool 43 being in contact with the surface of a depression 191 located on the top surface of the assay card 10, the depression 191 being aligned with a portion of the channel 16. In the embodiment shown, the tool 43 has a shape or size which permits it to penetrate the depression 191. It should be recognized that any suitable size and shape of the tool 43 may be employed.

FIG. 9(c) illustrates the assay card 10 being deformed by the tool 43 applying pressure to the assay card 10. In the embodiment shown, the tool 43 is positioned directly over the depression 191 of the channel 16, and the motion of the tool 43 (shown as arrow A) is perpendicular to the surface 42 of the assay card 10. In the embodiment shown in FIG. 9(c), the assay card 10 is deformed such that plastic of the substrate 41 fully obstructs the channel 16. As shown in FIG. 9(d), the deformed plastic of the substrate 41 is allowed to cool such that the obstruction of the channel 16 is maintained, FIG. 9(d) illustrates in dotted line the hidden portion of the channel 16 that has not been deformed and obstructed by the tool 43.

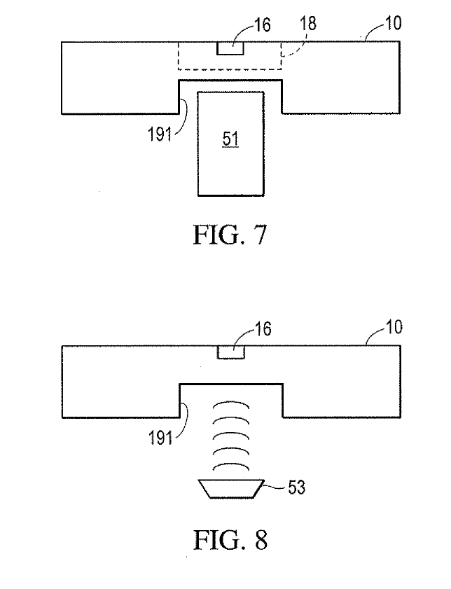

There are various other ways that heat may be applied to the substrate. For example, in an embodiment, the heating step includes applying a source of ultrasonic energy. FIG. 7 is a side view that illustrates schematically a method of applying heat to the substrate by applying a source of ultrasonic energy. In the embodiment shown, the source of ultrasonic energy consists of an ultrasonic horn 51. For example, FIG. 7 illustrates the assay card 10, having the channel 16 in communication with the reaction chamber 18. FIG. 7 illustrates the assay card 10 being heated by the ultrasonic horn 51, the ultrasonic horn 51 being positioned adjacent to the assay card 10. More specifically, FIG. 7 illustrates the assay card 10 being heated by the ultrasonic horn 51 wherein the ultrasonic horn 51 is positioned within, or in close proximity to, a depression 191 formed in the assay card 10. Such a depression 191 may enable the region of the assay card 10 that includes the channel 16 to be heated by a tool that is positioned relative to the assay card 10 on a side of the assay card 10 that is opposite from the side on which the channel 16 is located, thereby reducing the amount of heat that is required in order to heat the region of the assay card 10 that includes the channel 16. It should be recognized that the tool, e.g., the source of ultrasonic energy 51, may heat the assay card 10 with or without actually touching the assay card 10.

Additionally or alternatively, the heating step may include applying heat to the substrate with a light or laser beam, or other source of radiant energy. FIG. 8 illustrates the assay card 10 being heated by a radiant energy source 53 wherein the radiant energy source 53 is positioned within, or in close proximity to, a depression 191 formed in the assay card 10. As described above, such a depression 191 may enable the region of the assay card 10 that includes the channel 16 to be heated by a radiant energy source 53 that is located on a side of the assay card 10 that is opposite from the side on which the channel 16 is located, thereby reducing the amount of heat that is required in order to heat the region of the assay card 10 that includes the channel 16. Again, it should be recognized that the radiant energy source 53 may heat the assay card 10 with or without actually touching the assay card 10. Additionally or alternatively, the heating step may include applying heat to the substrate with a heated air jet. In accordance with an embodiment, electromagnetic induction may be employed to generate heat in just the tip of a tool 43. The use of electromagnetic induction may be advantageous in that the tool may be heated very quickly and, upon the removal of the electrical current to the inductor, the tool may be quickly cooled. Furthermore, by generating beat in just the tip of a tool 43, the amount of heat that is employed during the process may be reduced, thereby improving the safety and reliability of the process. Specifically, the use of less heat may enable an arrangement that reduces the likelihood of unintentionally heating a sample in the reaction chamber or inadvertently softening or deforming any portions of the substrate 41 that are desired to remain intact.

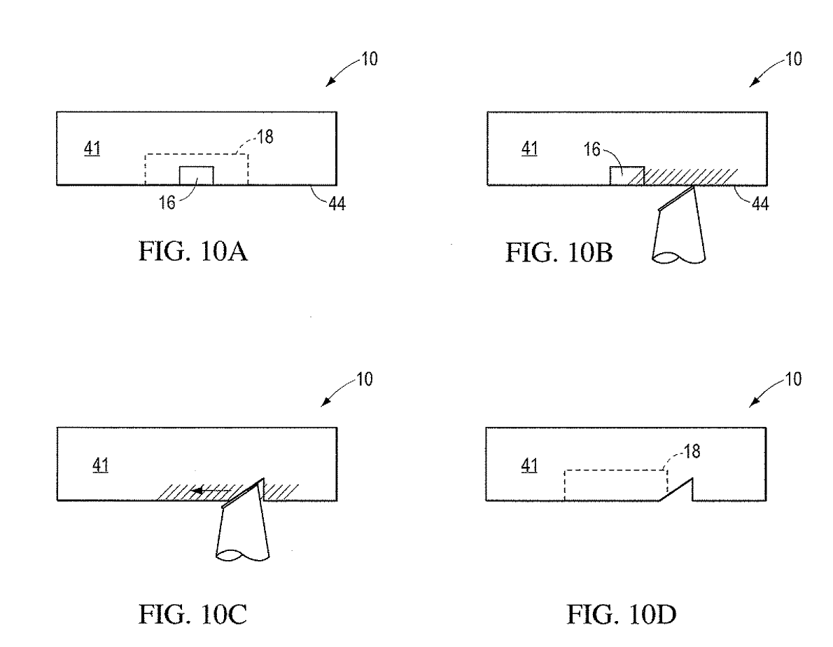

FIGS. 10(a) through 10(d) illustrate schematically a further embodiment of the invention in which a reaction chamber 18 is isolated by heating and deforming an assay card 10 with a heated tool in a region of the substrate 41 that is not aligned with a channel 16 to be obstructed. For example, FIG. 10(a) illustrates the assay card 10, having the channel 16 in communication with the reaction chamber 18. FIG. 10(b) illustrates the assay card 10 being heated by a tool 43 that contacts the assay card 10 in a region of the substrate 41 that is not aligned with, i.e., is offset relative to, the channel 16. Alternatively, the tool 43 may heat the assay card 10 without actually touching the assay card 10.

Again, the tool 43 may be any suitable shape or size, examples of which are described in further detail below. Also, the temperature at which the tool 43 may be heated and the duration of contact between the tool 43 and the assay card 10 may be varied and may be predetermined or determined by an operator during the heating process. Advantageously, the assay card 10, at least in a region of the channel 16, is heated to a predetermined temperature that is, for example, at least a softening temperature of the substrate 41 in a region that includes at least some portion of the substrate immediately adjacent to the channel 16. The heating of the assay card 10 in this manner may facilitate the deformation of the channel 16.

FIG. 10(c) illustrates the assay card 10 being deformed by the tool 43 applying pressure to the assay card 10. In the embodiment shown, the tool 43 is positioned to the side of the channel 16, and the shape of the tool 43 in addition to the motion of the tool 43 (shown as arrow A) are such that some portion of the heated substrate is caused to be displaced, i.e., is pushed into, the channel 16. It should be apparent that the position of contact of the tool 43 relative to the channel 16, the direction of motion of the tool and the amount of pressure applied may be varied to achieve suitable deformation of the assay card in the region of channel 16. In the embodiment shown in FIG. 10(c), the assay card 10 is deformed such that plastic of the substrate 41 fully obstructs the channel 16. As shown in FIG. 10(d), the deformed plastic of the substrate 41 may be allowed to cool such that the obstruction of the channel 16 is maintained and the reaction chamber 18 remains isolated.

Additionally or alternatively, heat may be applied to at least a portion of the assay card 10 on a second surface of the substrate, i.e., a surface to which the channel 16 is directly or most nearly adjacent. Such an arrangement is illustrated in, e.g., FIGS. 6(a) through 6(d), as set forth more fully above. Again, it should be apparent that, while FIGS. 6(a) through 6(d) and FIGS. 9(a) through 9(d) both illustrate the channel 16 being located adjacent to a bottom surface of the assay card 10, the channel 16 may instead be located adjacent to a top surface of the assay card 10, or may be located at any position between the top and bottom surfaces, so long as its location enables it to be heated and/or deformed in accordance with the devices and methods described herein.

As set forth above, there are various ways that the heated assay card 10 may be deformed such that plastic of the substrate 41 at least partially obstructs the channel 16. Various ones of the figures described hereinabove, e.g., FIGS. 6(a) through 6(d) and FIGS. 9(a) through 9(d), illustrate schematically a method of isolating a reaction chamber 18 by heating and deforming an assay card 10 with a single tool 43 that both heats and applies pressure to the assay card 10 for displacing at least some of the heated plastic. It should be apparent that any number of different tools may be employed to heat and/or deform the assay card 10. Also, a heated assay card 10 may be deformed without using a tool (e.g., by gravity), by using a tool that may or may not be the same tool that is employed to heat the assay card 10, or by using any one or more different tools configured to contact and/or apply pressure to the assay card 10.

It should also be apparent that varying amounts of contact and/or pressure (including no contact and/or pressure) may be employed depending on various factors, e.g., the type or grade of plastic used, degree of softening of the plastic, the size and shape of the channel 16, the size and shape of the tool 43, in addition to other factors. It should also be apparent that the relative positions of the tool 43 and the assay card 10 may also be varied, depending on such factors. Moreover, the heated assay card 10, or at least a portion of it may be deformed by the application of contact and/or pressure to the surface of the substrate to which the channel 16 is directly or most nearly adjacent, or, additionally or alternatively, to the opposite surface of the substrate.

Still further, the heated assay card 10, or at least a portion of the assay card 10, may be deformed by methods that do not require application of contact and/or pressure to a surface of the substrate. For example, the assay card 10, or at least a portion of the assay card 10, may be deformed by the application of a surface tension on the softened region of the substrate. Additionally or alternatively, the assay card 10, or at least a portion of the assay card 10, may be deformed by permitting gravity to deform the softened region of the assay card 10. Also, the assay card 10, or at least a portion of the assay card 10, may be deformed by the application of a pneumatic pressure or vacuum to the substrate. Furthermore, the assay card 10, or at least a portion of the assay card 10, may be deformed by moving the assay card so as to cause an inertial stress on the substrate, thereby deforming the softened plastic.

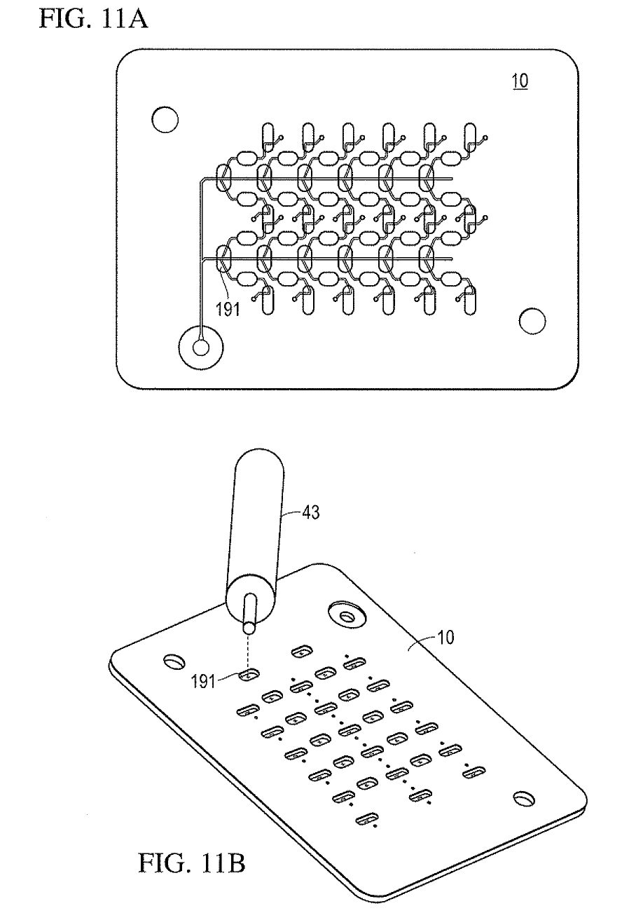

The invention has been described hereinabove as having various arrangements in which a tool 43, is employed to heat and deform a region of the assay card 10 to isolate a single reaction chamber 18. Alternatively, the invention may employ an arrangement in which each one of one or more tools may be employed to heat and deform a region of the substrate that includes two or more channels. In this manner, a single tool may be employed to isolate more than one reaction chamber. FIGS. 11(a) and 11(b) illustrate such an arrangement. Specifically, FIG. 11(a) illustrates an assay card 10 in which a plurality of depressions 191 are formed. Each one of these depressions 191 may be sized and shaped and located so as to be aligned with more than one of the channels 16. Specifically, and as shown in FIG. 11(a), each one of the depressions 191 may have an oval shape, the interior of which is aligned with each one of two channels 16 that lead into respective reaction chambers 18.

Each one of these depressions 191 may be configured to receive a tool, such as shown in FIG. 11(b). Advantageously, each tool is configured such that, when it is employed to heat and/or deform the region of the assay card 10 adjacent to its respective depression 191, the tool causes heating of and at least partial obstruction, and preferably complete obstruction of, each one of the two channels 16. It should be recognized that the invention may employ an arrangement in which a tool may align with, or may otherwise be configured to heat and/or deform any number of channels so as to isolate any number of respective chambers.

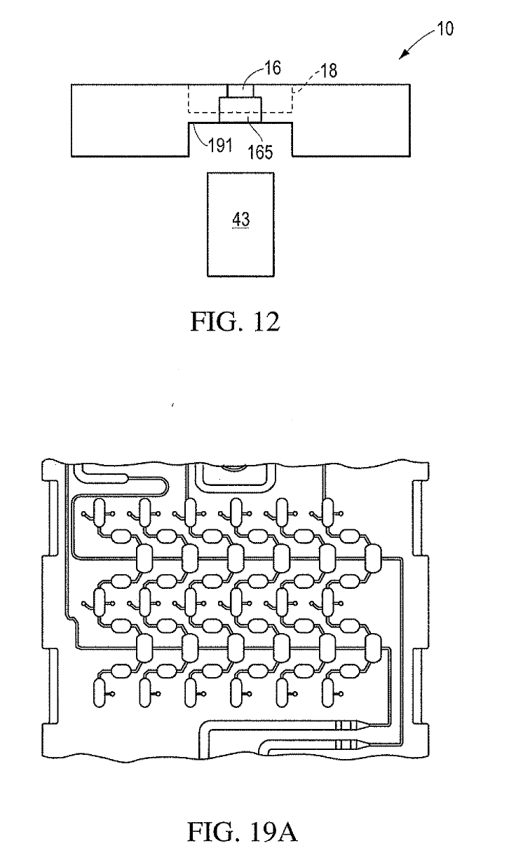

While various embodiments set forth hereinabove describe the substrate 41 being formed of a single type of plastic, the invention includes embodiments in which the substrate 41 is formed of more than one type of plastic. For example, FIG. 12 is a side view that illustrates schematically a method of applying heat to the substrate with a heated tool 43, wherein the substrate 41 is formed of more than one type of plastic, e.g., by co-molding. FIG. 12 illustrates the assay card 10 having channel 16 in communication with reaction chamber 18. FIG. 12 illustrates the assay card 10 being formed primarily of a first material, e.g., a first type of plastic, and having a region 165 adjacent to the channel 16 that is formed of a second material, e.g., a second plastic, that is different from the first material. More specifically, FIG. 12 illustrates an arrangement in which the assay card 10 may be heated by the tool 43 wherein the tool 43 is positioned within, or in close proximity to, a depression 191 of the assay card 10 that is defined in the assay card 10, the second material being disposed between the channel 16 and the tool 43. In an embodiment, the softening temperature of the first material may be greater than the softening temperature of the second material 165. This may enable the second material 165, which is within the region of the assay card 10 that includes the channel 16, to be melted or otherwise heated to a temperature suitable for it to be deformed without the first material reaching its softening temperature. Such an arrangement may decrease the likelihood that the portions of the substrate 41 that are formed of the first material are inadvertently melted or deformed. A further decrease in such a likelihood may be accomplished with the arrangement shown, e.g., by employing a depression 191 and having the tool act on the side of the assay card 10 that is opposite from the side on which the channel 16 is located. Again such an arrangement may reduce the amount of heat that is required in order to heat the region of the assay card 10 that includes the channel 16, and thus may reduce the likelihood of unintentionally heating a sample in the reaction chamber or inadvertently softening or deforming any portions of the substrate 41 that are desired to remain intact. It should be apparent that the region 165 may be positioned relative to the channel 16 in any suitable location so as to be heated and/or deformed in any of the manners described hereinabove.

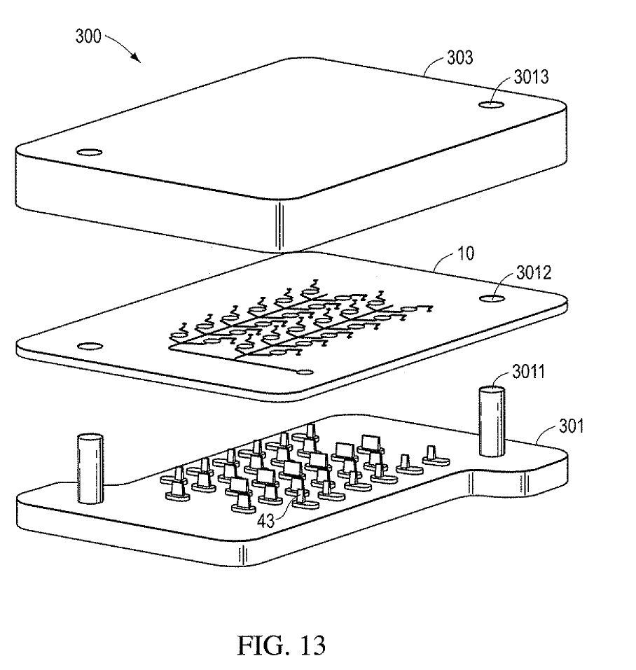

The assay card of the invention, in accordance with any of the various embodiments described hereinabove, may be configured for use in a system. For example, FIG. 13 is an exploded perspective view of a system 300 that includes a support device 303. The support device 303 includes one or more bores 3013. The system 300 also includes the assay card 10 as set forth in any of the previously-described embodiments, the assay card 10 further including one or more bores 3012. The system 300 further includes a tool device 301. The tool device 301 includes one or posts 3011. In addition, the tool device 301 includes one or more tools 43 that may be arranged in any suitable configuration. The posts 3011 of the tool device are configured to be inserted in the bores 3012 of the assay card 10 and the bores 3013 of the support device 303 in order to facilitate the alignment of the tools 43 with respective portions of the assay card 10, e.g., the channels 16 to be obstructed. Of course, any suitable arrangement of these, or other components that may operate to isolate chambers of an assay card as described hereinabove, may be employed in such a system.

Isolating the reaction chambers 18 may accomplish a number of desirable objectives, including prevention of cross-contamination of the reactions in the respective reaction chambers by preventing diffusion of reactants from one chamber to another through the channels, and preclusion of air bubbles from entering the reaction chambers. In the assay card and the method of the invention, chamber isolation can be accomplished safely, accurately and reliably. For example, as set forth above, the assay card 10 may be heated by a device, e.g., a heated tool 43 or an ultrasonic horn 51, that is positioned within, or in close proximity to, depressions 191 that are defined in the assay card 10. Such depressions 191 enable the region of the assay card 10 that includes the channel 16 to be heated by a tool that is located on a side of the assay card 10 that is opposite the side on which the channel 16 is located. Such an arrangement may reduce the amount of heat that is required in order to heat the region of the assay card 10 that includes the channel 16, and may reduce the likelihood of unintentionally heating a sample in the reaction chamber or inadvertently softening or deforming any portions of the substrate 41 that are desired to remain intact.

Additional advantages are obtained through use of a tool to heat and deform a region of the substrate that includes two or more channels. Specifically, and as illustrated for example in FIG. 11(a), the assay card of the invention may include a region, e.g., the depression 191, which may be sized and shaped for receiving a tool that is aligned with more than one channel 16. By aligning a tool with two or more channels 16, multiple reactions chambers 18 may be isolated with a single tool. As a result, the number of tools that may be needed to isolate a given number of reactions chambers 18 may be reduced, thereby decreasing the cost and complexity as compared to other assay card systems and methods.

Further advantages are obtained where the tool device include a plurality of tools, each one of which may be employed to heat and deform a region of the substrate that includes a channel. Specifically, and as illustrated for example in FIG. 12, the assay card of the invention may include a plurality of tools 43, each one of which may be sized and shaped so as to be aligned with a channel 16 (or more than one channel 16). By employing a tool device that includes a plurality of tools, each one of which may be employed to heat and deform a region of the substrate that includes a channel, multiple reactions chambers 18 may be isolated with a single application of the tool device. As a result, the number of steps that may be needed to isolate a given number of reactions chambers 18 may be reduced, thereby decreasing the cost and complexity as compared to other assay card systems and methods.

Still further, the assay card of the invention, may provide advantages by virtue of the substrate 41 being formed of more than one type of material, e.g., by co-molding. Specifically, and as illustrated for example in FIG. 13, the assay card of the invention may be formed in a first region of a first material, e.g., a first type of plastic, and formed in a second region, e.g., a region that is directly adjacent to a channel, of a second material, e.g., a second plastic, that is different from the first material. In this manner, the softening temperature of the first material may be greater than the softening temperature of the second material 165. This may enable the second material 165, which is within the region of the assay card 10 that includes the channel 16, to be melted or otherwise heated to a temperature suitable for it to be deformed without the first material reaching its softening temperature. The use of less heat may enable an arrangement that reduces the likelihood of unintentionally heating a sample in the reaction chamber or inadvertently softening or deforming any portions of the substrate 41 that are desired to remain intact.

### EXAMPLE

The method of the invention, in accordance with an embodiment thereof, was tested with an assay card 100 such as that shown in FIGS. 4(a) and (b). More specifically, a PSA lined film 144 with an aluminum foil carrier was laminated on a molded COP substrate 141, and the assembled COP assay card 100 was set up as shown in FIG. 13. Then, the three components shown in FIG. 13 were placed between two flat platen in a Carver Press, where the platen on the side adjacent to the tool device 301 was heated up to 200° C., while the other platen was kept at room temperature. The two platen were clamped until the entire surface area of the support device 303 and the tool device 301 were in contact with their respective adjacent platens. The temperature of the tools 43 were monitored by a thermocouple being glued on the tool 43. When the temperature of the tool 43 reached approximately 200° C., the two platen were further clamped to a pressure of about 150 lbs. for about 5 seconds, Then the clamping force was quickly released. After this procedure was completed, the PSA lined film 144 was de-laminated in order to inspect the results. It was observed that the COP substrate 141 was deformed.

In addition, the above-described test was also performed using various conditions, e.g., temperatures ranging from 180° C. to 250° C. and pressures ranging from about 50 to 150 lbs. In all of these tests, results similar to the result of the above-described test were obtained.

In order to determine whether, and thereby minimize the likelihood that, the high temperature of the tool 43 will be undesirably conducted to an analyte sample located in a reaction chamber 18, further testing was carried out to measure the temperature in a reaction chamber 18 during the application of the tool. A thermocouple (Omega Engineering, model 5SRTC-TT-T-40-36) was embedded in a reaction chamber 18 with a thermally conductive epoxy (Omega Engineering, model OB 200). A soldering iron having a surface temperature of about 300° C. was manually pushed against the channel region of the COP substrate 141 for 3 seconds and the temperature reading from the thermocouple was monitored. The measured temperature was determined to be dependent upon the location of the tool 43 relative to the reaction chamber 18, and ranged from 24° C. to 38° C. The temperature measurement results indicate that the method of the invention would not adversely affect an analyte sample and the polymerase chain reaction (PCR) mix or the reverse-transciptase polymerase chain reaction mixture (RT-PCR) in the reaction chamber 18.

Numerical calculation data was collected in order to describe temperatures in the reaction chambers 18 when tools 43 having a temperature of 200° C. were contacted for 2.5 seconds, the tools 43 being contacted with an assay card 100 that included depressions 191 having a depth of 500 μm. It was determined that the maximum temperature in the reaction chamber 18 was about 34° C. The numerical calculation confirmed again that the hot temperature on the tool 43 would not adversely affect an analyte sample and the PCR mix in the reaction chamber 18.

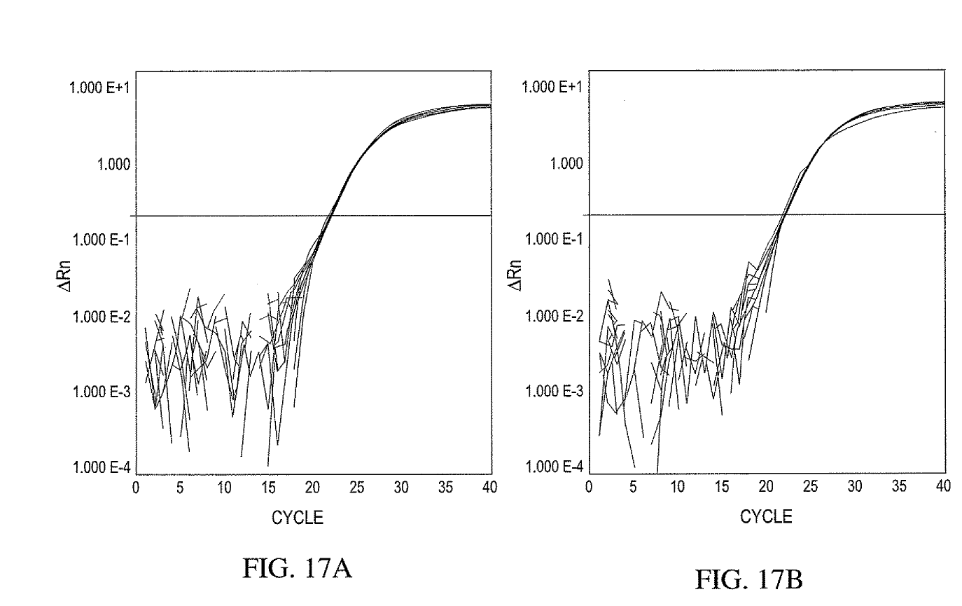

After the above-described test was performed, DNA amplification by real-time PCR was performed in an assay card 100. A sample of Streptococcus pneumoniae (concentration of 10 k copies/uL) was provided in all 24 chambers of an assay card 100. Then, the channels 16 and the vent channels 20 were occluded by applying a soldering iron having a surface temperature of about 300° C. For comparison purpose, as tool at room temperature was applied to another assay card prepared with the same target sample. DNA amplification by real-time PCR was performed in an Applied Biosystems 7900HT Real-Time PCR System for the two assay cards. FIGS. 17(a) and (b) are graphs that illustrate the Delta Rn curves. The control assay card, the results for which are illustrated in FIG. 17(a), showed an average Ct value of 22.10 with standard deviation of 0.16. In contrast, the assay card 100 to which heat was applied in accordance with the invention, the results for which are illustrated in FIG. 17(b), showed an average Ct value of 22.30 with the standard deviation of 0.22. A series of PCR runs showed the same result, namely that the method of the present invention, in accordance with various embodiments described hereinabove, are suitable for reaction chamber isolation.

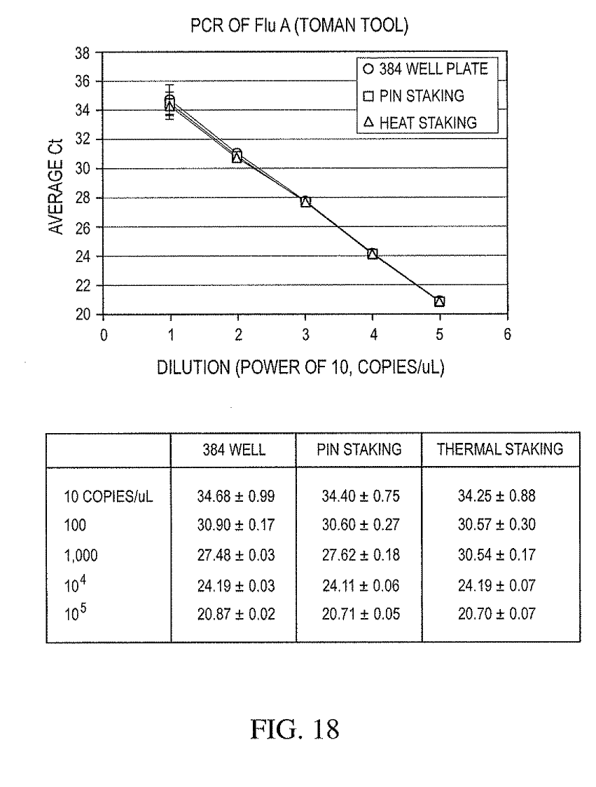

DNA amplification by real-time PCR including reverse-transcriptase (RT) at 48° C. was also performed in an assay card 100. A sample of Flu A was provided in all 24 wells of an assay card 100. Then, the channels 16 and the vent channels 20 were occluded by applying a thermal isolation with a thermal isolation teeth temperature of about 240° C. For comparison purpose, a tool at room temperature was applied to another assay card prepared with the same target sample. DNA amplification by real-time PCR was performed in an Applied Biosystems 7900HT Real-Time PCR System. 5 different concentrations of Flu A samples were amplified in 5 different assay cards. FIG. 18 illustrates a graph that illustrates Ct value variation as a function of the concentration. For control and comparison, the Ct values from room-temperature well isolation methods and from 384-well plate PCR are also shown in the graph. The fact that all the Ct values collapse onto a single curve implies that the thermal isolation applied onto the assay card does not affect the RT step and that the method of the present invention, in accordance with various embodiments described hereinabove, are suitable for reaction chamber isolation.

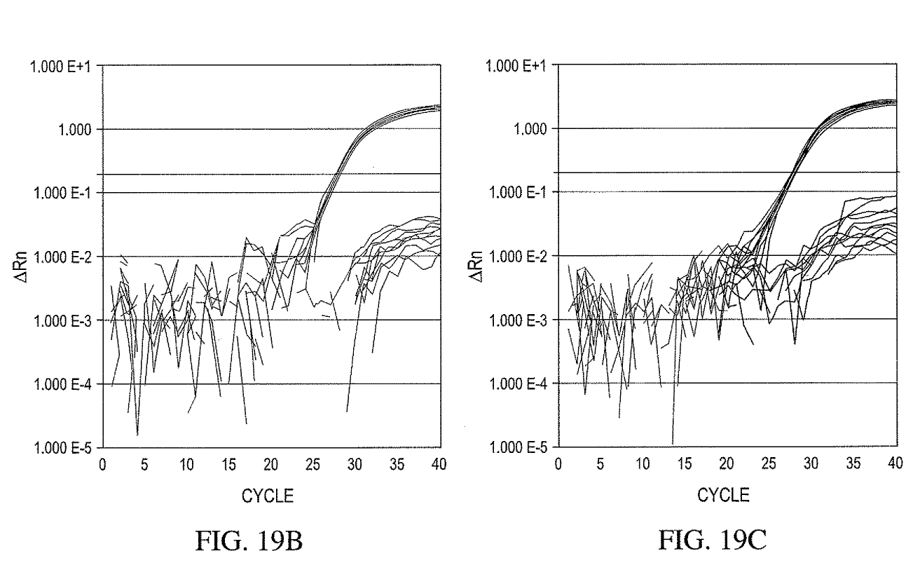

FIG. 19 described the PCR experiment in an assay card 100 with dried reagents. Two different assays (primers, probes and enzymes for Flu B and for Mycoplasma pneumoniae, respectively) were spotted in a checkerboard pattern as shown in FIG. 19(a). A sample was Flu B with a concentration of 1 k copies/μL was provided in all 24 wells of assay card 100. Then the channels 16 and the vent chamber 20 were occluded by applying a thermal isolation with a thermal isolation teeth temperature of about 240° C. For comparison purpose, a tool at room temperature was applied to another assay card prepared with the same target sample DNA amplification by real-time PCR was performed in an Applied Biosystems 7900HT Real-Time PCR System for the two assay cards. FIGS. 19(a) and (b) are graphs that illustrate the Delta Rn curves. While the amplifications were successful in the wells for Flu B, no amplification was detected in the wells for Mycoplasma pneumoniae, indicating the thermal isolation method successfully prevents the cross-contamination among the wells.

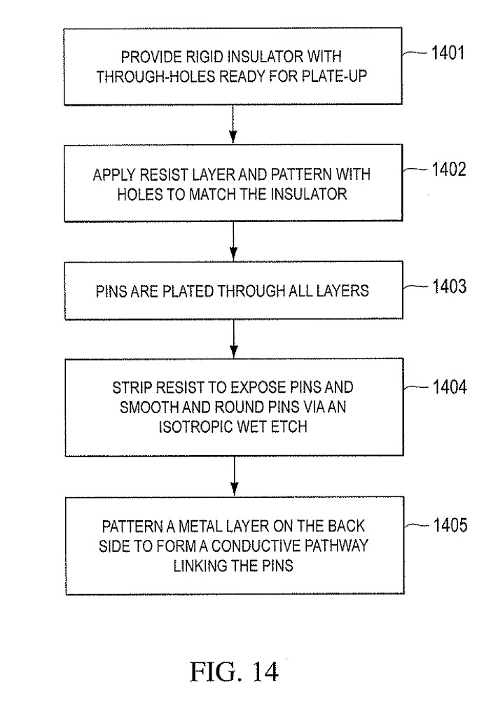

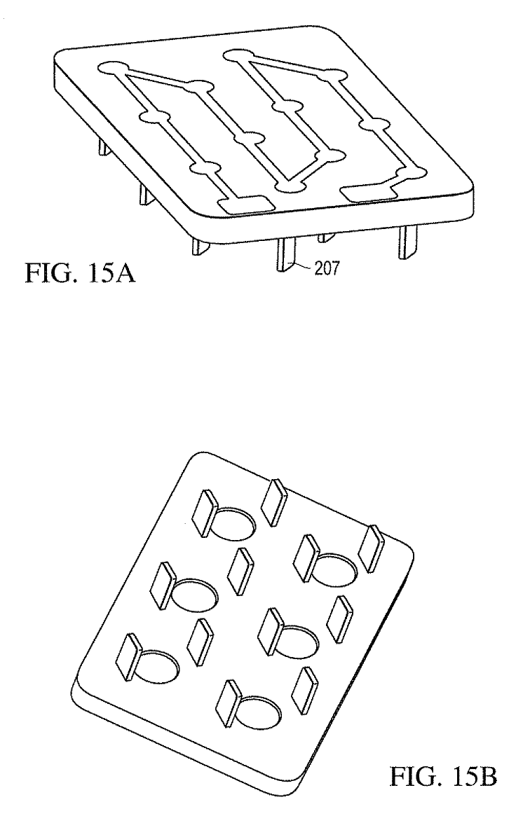

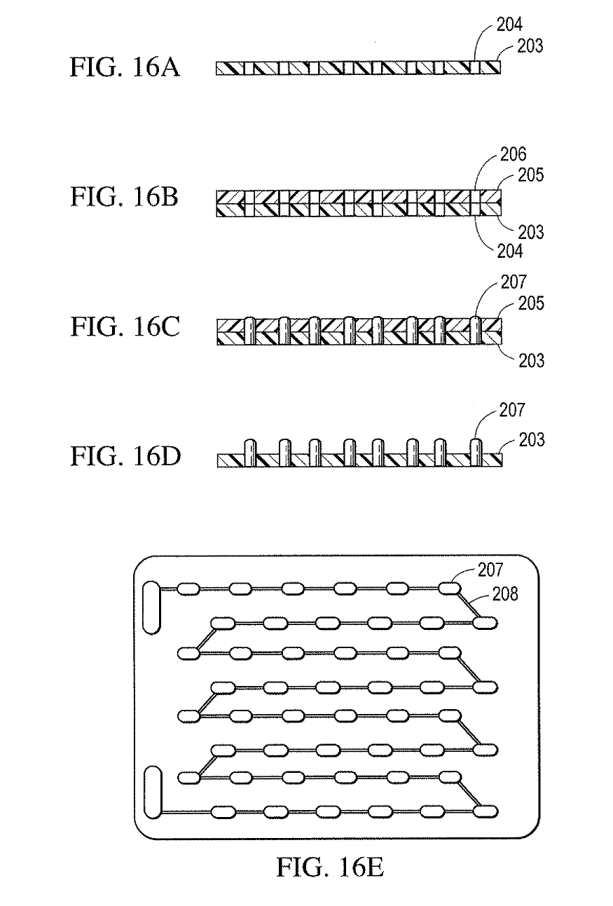

The invention, according to various embodiments thereof also relates to a method of manufacturing or fabricating a tool device. FIG. 14 is a flowchart that illustrates the steps that may be employed to manufacture or fabricate the tool device, e.g., the hot staker head 201, illustrated in FIGS. 15(a) and (b), which are described more fully below. Referring to FIG. 14, at step 1401 the manufacture or fabrication of the tool device, e.g., the hot staker head 201, may include the step of providing an insulator, which may be a rigid insulator, with through-holes ready for plate-up. FIG. 16(a) is a side cross-sectional view that illustrates such a rigid insulator 203, with through-holes 204 ready for plate-up.

At step 1402 of the flowchart illustrated in FIG. 14, a resist layer is applied. FIG. 16(b) is a side cross-sectional view that illustrates a resist layer 205 being applied to the rigid insulator 203. In addition, at step 1402, the resist layer is patterned with holes which match the holes of the insulator, As shown in FIG. 16(b), the resist layer 205 is patterned with holes 206 which match the holes 204 of the rigid insulator 203.

At step 1403 of the flowchart illustrated in FIG. 14, pins are plated through all layers. FIG. 16(c) is a side cross-sectional view that illustrates pins 207 being plated through all layers, e.g., through the resist layer 205 and the rigid insulator 203. At step 1404 of the flowchart illustrated in FIG. 14, the resist layer is stripped to expose the pins. FIG. 16(d) is a side cross-sectional view that illustrates the resist layer 205 being stripped so as to expose the pins 207. In addition, at step 1404, an isotropic wet etch process may be performed so as to smooth and round the pins 207.

At step 1405 of the flowchart illustrated in FIG. 14, a metal layer on the back side of the device is patterned so as to form a conductive pathway linking the pins 207. An example of such a pattern 208 is illustrated in FIG. 16(e), which provides a bottom view of a hot staker head formed by the above-described method. It should be recognized that any suitable pattern may be employed in order to form a conductive pathway linking the pins 207.

Referring now to FIGS. 15(a) and (b), there are provided perspective top and bottom views, respectively, of a tool device, e.g., a hot staker head 201, formed in accordance with the steps set forth in the flowchart of FIG. 14. It should be recognized that any arrangement of the pins 207 may be employed, the pins 207 forming and functioning as, e.g., the heated tools 43, described in the various methods above. In an embodiment, a rigid thermal and electrical insulating base (e.g., ceramic) may be used as a base to form the array of pins 207 that address the points to be sealed on the assay cards. Referring to FIG. 15(a), the hot staker pins 207 may be connected together into a resistive heater element designed to include edge connector capability.

The method of manufacturing or fabricating the tool device as set forth above, and the use of same in a method for isolating chambers of an assay card, may provide advantages as compared to conventional manufacturing and fabrication methods. Again, by introducing heat into the staking process, excessive downward forces on the assay card may be greatly reduced or avoided. Furthermore, the amount of heat that is employed to melt and/or deform the substrate material is very localized. Still further, the invention may, in accordance with various embodiments described herein, reduce the thermal mass of any heated surface thus reducing the potential to introduce heat to sensitive areas of the assay card.

Thus, the several aforementioned objects and advantages of the invention are most effectively attained. Those skilled in the art will appreciate that numerous modifications of the exemplary embodiment described hereinabove may be made without departing from the spirit and scope of the invention. For example, the invention may be employed in other biochemical assays, such as isothermal amplification, clinical chemistry assays, and others. Although various exemplary embodiments of the invention have been described and disclosed in detail herein, it should be understood that this invention is in no sense limited thereby.

What is claimed is:

1. A method for isolating chambers on an assay card, the assay card comprising a substrate formed of plastic having a softening temperature, the substrate defining a first channel in communication with a first chamber and a second channel in communication with a second chamber, the method comprising the steps of:
heating the assay card in a region of the first and second channels to at least the softening temperature;
simultaneously deforming, with a single tool, the assay card in the region of the first and second channels such that plastic of the substrate is caused to at least partially obstruct both the first and second channels.

2. The method of claim 1, further comprising the step of cooling the deformed plastic.

3. The method of claim 1, wherein the heating step includes at least one of: contacting the substrate with the tool, the tool being heated; applying a source of ultrasonic energy to the substrate; applying heat to the substrate with one of a light or laser beam; and applying heat to the substrate with a heated air jet.

4. The method of claim 3, wherein contacting the substrate with a heated tool includes at least one of inserting the tool into the channel; and contacting a region of the substrate adjacent to the channel.

5. The method of claim 1, wherein the deforming step includes at least one of:
application of a surface tension on the softened region of the substrate; permitting gravity to deform the softened region of the substrate; application of a pneumatic pressure or vacuum to the substrate; and moving the assay card so as to cause an inertial stress on the substrate.

6. The method of claim 1, wherein the heating and deforming steps include heating and deforming the assay card from a side of the substrate to which the channels are directly or most nearly adjacent.

7. The method of claim 1, wherein the heating and deforming steps include heating and deforming the assay card from a side of the substrate that is opposite the side of the substrate to which the channels are directly or most nearly adjacent.

8. A method for isolating a chamber of an assay card, the assay card comprising a substrate formed of plastic having a softening temperature, the substrate defining the chamber and a channel in communication with the chamber, the substrate further defining a depression in a surface of the substrate, the depression being aligned with at least a portion of the channel, the method comprising the steps of:
heating the assay card in a region of the depression and the channel to at least the softening temperature; and
deforming the assay card in the region of the depression such that plastic of the substrate at least partially obstructs the channel.

9. The method of claim 8, further comprising the step of cooling the deformed plastic.

10. The method of claim 8, wherein the heating and deforming steps include contacting and applying pressure to a surface of the depression of the substrate with a heated tool.

11. The method of claim 8, wherein the heating step includes at least one of: applying a source of ultrasonic energy to a surface of the depression of the substrate; applying heat to a surface of the depression of the substrate with one of a light or laser beam; applying heat to the substrate with a heated air jet.

12. The method of claim 8, wherein the substrate defines two or more chambers and two or more channels, each chamber having a respective channel, and wherein the heating and deforming steps simultaneously heat and deform the assay card in a region that includes the two or more channels.

13. The method of claim 12, wherein the region that includes the two or more channels is simultaneously heated by contact with a single heated tool.

14. A method for isolating chambers of an assay card, the assay card comprising a substrate formed of a first material having a first softening temperature and a second material having a second softening temperature, the substrate defining a channel in communication with a chamber, the second material being adjacent to the channel, the method comprising the steps of:
heating the assay card in a region of the channel to at least the second softening temperature; and
deforming the assay card in the region of the channel such that the second material at least partially obstructs the channel.

15. The method of claim 14, wherein the first material is a first type of plastic and the second material is a second type or grade of plastic different from the first type of plastic.

16. The method of claim 14, wherein the first softening temperature is greater than the second softening temperature, and wherein the heating step includes heating the assay card in a region of the channel to a temperature that is greater than the second softening temperature but less than the first softening temperature.

17. The method of claim 14, wherein the channel is located adjacent to a first surface of the substrate, a second surface of the substrate being opposite the first surface and including a depression, and the depression is aligned with at least a portion of the channel, wherein the second material is disposed between respective bottom surfaces of the depression and the channel, such that, upon the second material being heated to at least the second softening temperature, the second material is deformable for obstructing the channel, and further comprising the step of cooling the deformed plastic, and wherein the heating and deforming steps include contacting and applying pressure to a surface of the depression of the substrate with a heated tool, and further comprising a cooling the deformed material.

18. An assay card comprising:
a substrate formed of plastic having a softening temperature, the substrate defining a chamber and a channel in communication with the chamber, the substrate further defining in a surface a depression that is aligned with at least a portion of the channel, wherein, upon heating the assay card in a region of the depression and the channel to at least the softening temperature, the plastic of the substrate is deformed so as to at least partially obstruct the channel.

19. The assay card of claim 18, wherein the substrate defines two or more chambers and two or more channels, each chamber having a respective channel, and wherein at least a portion of each of the two or more channels is positioned adjacent to the depression so as to be simultaneously heated and deformed, and wherein the deformed plastic is cooled so as to maintain the at least partial obstruction of the channels.

20. The assay card of claim 18, wherein the plastic includes a first region having a first softening temperature and a second region having a second softening temperature or a first material having a first softening temperature and a second material having a second softening temperature, and wherein the first softening temperature is greater than the second softening temperature.

21. A system comprising:
an assay card comprising a substrate formed of plastic having a softening temperature, the substrate defining a first channel in communication with a first chamber and a second channel in communication with a second chamber; and
a tool having a size and shape such that, upon the assay card being heated in a region of the first and second channels to at least the softening temperature, the tool simultaneously deforms the assay card in the region of the first and second channels such that plastic of the substrate is caused to at least partially obstruct both the first and second channels.

22. The system of claim 21, wherein the tool is heated and positioned relative to the assay card so as to at least one of: be inserted into the channel; and positioned relative to the assay card so as to contact a region of the substrate adjacent to the channel.

23. The system of claim 21, wherein the system includes at least one of: a source of ultrasonic energy for heating the assay card; a light or laser beam for heating the assay card; and a heated air jet for heating the assay card.

24. The system of claim 21, wherein the heated tool is positioned relative to the assay card so as to heat and deform the assay card from a side of the substrate to which the channels are directly or most nearly adjacent.

25. The system of claim 21, wherein the heated tool is positioned relative to the assay card so as to heat and deform the assay card from a side of the substrate that is opposite the side of the substrate to which the channels are directly or most nearly adjacent.
